(19) United States
(12) Patent Application Publication
(10) Pub. No.: US 2006/0089648 A1
(43) Pub. Date: Apr. 27, 2006

(54) Versatile bone plate systems particularly suited to minimally invasive surgical procedures

(76) Inventor: Michael A. Masini, Ann Arbor, MI (US)

Correspondence Address: GIFFORD, KRASS, GROH, SPRINKLE & CITKOWSKI, P.C, PO BOX 7021, TROY, MI, 48007-7021, US

(21) Appl. No.: 10/974,190

(22) Filed: Oct. 27, 2004

(51) Int. Cl. A61F 2/30

(52) U.S. Cl. 606/69

(57) ABSTRACT

A bone plate adapted for distal radius fixation and other indications includes a first plate portion including one or more fastener-receiving apertures, a second plate portion also including one or more fastener-receiving apertures, and a mechanism coupling the first and second plate portions. The mechanism facilitates a first state, wherein the plate portions may be articulated to achieve a desired angular orientation, and a second state, wherein the plates are rigidly locked into position at the desired angular orientation. Patentably distinct plate extensions, locking screws, and drill guides are also disclosed.

### FIELD OF THE INVENTION

This invention relates generally to bone plates and, in particular, to improved bone plates particularly suited to fractures of the distal radius.

### SUMMARY OF THE INVENTION

Fractures of the distal radius are one of the most common fractures, with over a third of a million occurring annually in the United States alone. Distal radius fractures account for 17% of all fractures treated in the emergency room. They are particularly prevalent in pediatric and in geriatric patients.

Different types of distal radius fractures exist, including Colles' fracture, Smith's fracture, and Barton's fracture. Colles' is a distal metaphysial fracture with dorsal displacement and angulation, radial angulation, and radial shortening. Smith's is a distal metaphyseal fracture with volar displacement and angulation. Barton's is a fracture-dislocation wherein the rim of the distal radius is displaced volarly or dorsally along with the distal carpus. This is different from Smith's or Colles' in that the dislocation is the primary indication, with the radial fracture noted secondarily.

A Colles' fracture often results from a fall on an outstretched hand, causing tension forces on the palmar radius and bending and compression forces on dorsal aspect of the radius. This incidence of this condition has increased due to the popularity of rollerblading, skateboarding, and other activities. Smith's fracture may be caused by a backward fall on the palm of an outstretched hand, causing pronation of the upper extremity while the hand is fixed to the ground.

Despite the high incidence of such fractures, they remain difficult to treat. There are many reasons for this, including the number of bones and bone fragments that are often involved, the need for angular fixation in multiple planes, and difficulties associated with providing requisite compression at the fracture site(s). Both external and internal devices are in use.

Colles' fractures may be treated with a dorsal or volar plate and screw system. Such plates are generally T-shaped, having a head and body portions with screw-receiving holes. Procedurally, the bone fragments are aligned and the body portion of the plate is screwed to an integral portion of the radius proximal of the fracture. Screws are then provided through the holes in the head portion to define a stabilizing framework about the fractured bone fragments heal.

FIG. 1 shows a typical dorsal fixation situation including a thin plate 102 secured to the dorsal side of the radius 106 with screws 108 on either side of a fracture site 104. FIG. 2 shows a conventional volar plate 206. FIGS. 3 and 4 are frontal views of typical plate designs used for distal radius fixation.

These existing designs have many shortcomings, including an inability to accomodate the complex, variable anatomy that is often involved with fractures of this type. Note in FIGS. 1 and 2, for example, that both volar and dorsal plates should not be flat, but rather, should include bends at regions 110 and 120, for enhanced conformity to angular variation in the distal radius, which vary from patient to patient and may be in the range of 0-22 degrees, with 11 degrees or thereabouts being typical. Additionally, although newer plating system may take advantage of polyaxial and locking screws, breakage can often occur at the screw location due to the low profile of typical plates.

### SUMMARY OF TH INVENTION

This invention resides a bone plate adapted for distal radius fixation and other indications. The preferred embodiment comprises a first plate portion including one or more fastener-receiving apertures, a second plate portion also including one or more fastener-receiving apertures, and a lockable hinge mechanism coupling the first and second plate portions. The mechanism facilitates a first state, wherein the plate portions may be articulated to achieve a desired angular orientation, and a second state, wherein the plates are rigidly locked into position at the desired angular orientation. Patentably distinct plate extensions, locking screws, and drill guides are also disclosed.

### BRIEF DESCRIPTION OF THE DRAWINGS

FIG. 1 is a side-view drawing of the typical prior-art dorsal plating system used to set fractures of the distal radius;

FIG. 2 is a drawing of a typical volar plate;

FIG. 3 is a top-view of a prior-art t-shaped plate for distal fracture repair;

FIG. 4 is a top-view of a prior-art bone plate suitable to distal radius fracture repair, including fork-like tines;

FIG. 5 is a side-view drawing of an articulating, locking bone plate according to the invention;

FIG. 6 is a drawing which shows how the present invention may accommodate multiple articulations in different planes;

FIG. 7A is a perspective view of one articulation/locking system according to the invention;

FIG. 7B shows how a portion of plates according to the invention may be curved for greater strength and/or conformity to bone;

FIG. 9 is a side-view, simplified drawing, which shows a different type of articulation and locking system of the invention;

FIG. 10 is a side-view drawing and partial cross-section illustrating a patentably distinct bone plate screw sleeve lengthening to enhance the structural integrity;

FIG. 11A is a side-view drawing and partial cross-section showing a further patentably distinct concept involving a metal-metal taper locking system;

FIG. 11B is a side-view drawing and partial cross-section showing a further patentably distinct concept involving a metal-metal taper locking system and multiple threaded areas;

FIG. 12A is a side-view drawing and partial cross-section showing a yet a different, patentably distinct concept involving a metal-metal taper locking system;

FIG. 12B is a side-view drawing and partial cross-section showing a yet a different, patentably distinct concept involving a metal-metal taper locking system and multiple threaded areas;

FIG. 12C shows how a tab may be used as opposed to a full set of threads;

FIG. 13A is an A-P view of an alternative articulation/locking system according to the invention;

FIG. 13B is a lateral view of an alternative articulation/locking system according to the invention;

FIG. 14A is an A-P view of a further articulation/locking system according to the invention;

FIG. 14B is a lateral view of a further alternative articulation/locking system according to the invention;

FIG. 15 illustrates a drill guide according to the invention;

FIG. 16 illustrates a patentably distinct stepped drill according to the invention;

FIG. 17 illustrates a patentably distinct stepped screw according to the invention; and

FIG. 17 illustrates a patentably distinct polyaxial locking screw according to the invention

### DETAILED DESCRIPTION OF THE PREFERRED EMBODIMENTS

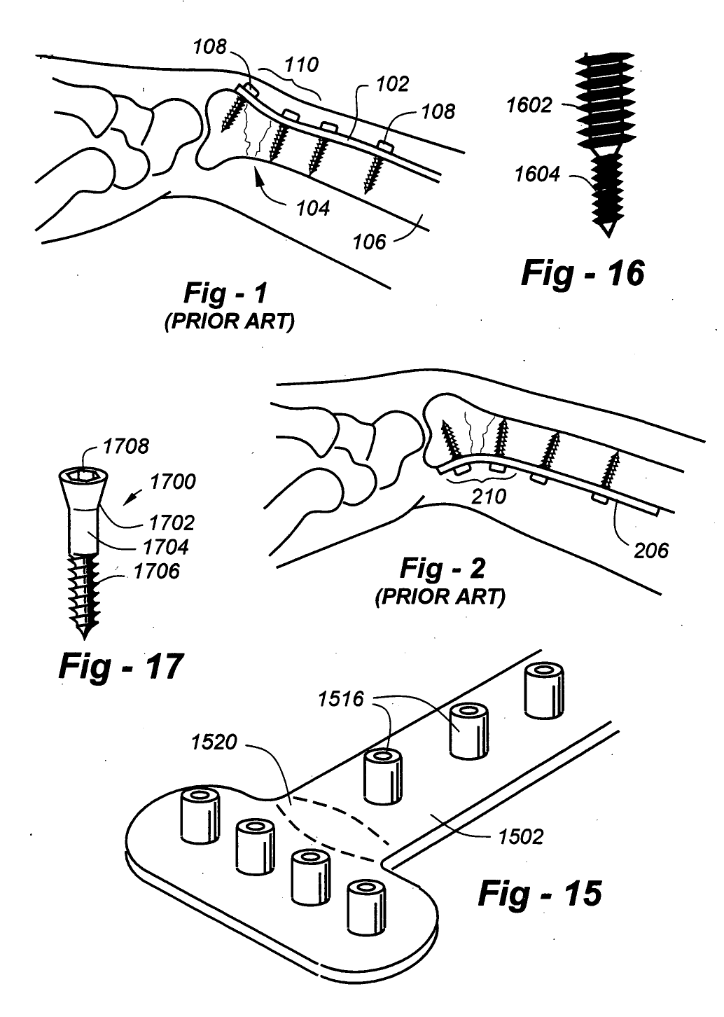

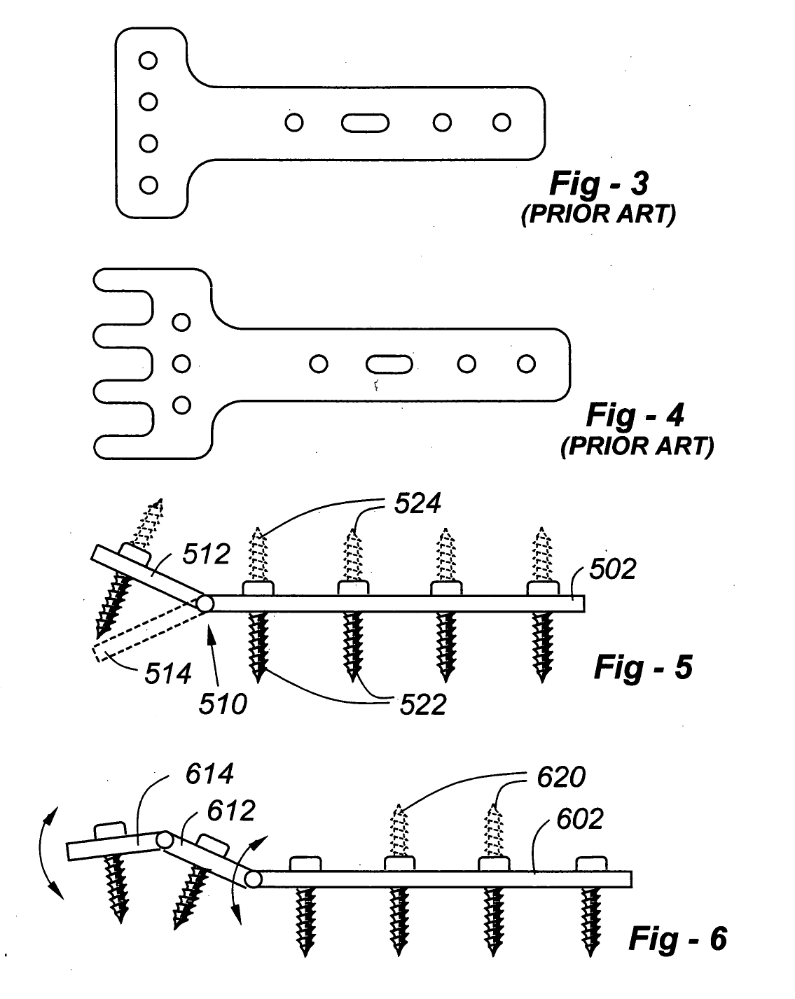

Having discussed the prior art with reference to FIGS. 1 through 4, the reader's attention is now directed to FIG. 5, which illustrates the broad concept of an articulating/locking bone plate system according to the invention. The system includes a first plate portion 502, which is coupled to a second plate portion 512 through a hinge 510, allowing angular variation between 502 and 512 to different planes, such as 514. Both the portion 502 and 512 include one or more screw- or pin-receiving apertures, enabling screws to penetrate therethrough in either direction, as indicated by 522 and 524.

In all embodiments, the area of articulation, indicated at 510 in FIG. 5, may be initially adjustable to suit a particular patient physiology, after which time the interrelationship between the portions 502 and 512, for example, may be locked in position. Accordingly, such a versatile system may have numerous applications, including the ability to internally fix distal radius fractures from both the volar and dorsal approach. Importantly, the invention also allows fixation of bone fragments independently, followed by adjustments of the plate system(s) to improve alignment following fixation.

The materials used for this and other devices described herein may be any suitable biocompatible material, such as metal, ceramic, metal/ceramic, hard plastics, and so forth. Bony in-growth and/or on-growth surfaces may be used, if desired, as well as bio-resorbable components. Nor do any of the embodiments described herein preclude the use of poly-axial screws, locking screws, or slots, as opposed to mound apertures to facilitate compression at the fracture site(s). Further, although the invention is described with reference to bone plate systems particularly suited to distal radial fractures, those of skill will recognize that the concepts disclosed herein are equally applicable to traditional screw-plate systems.

FIG. 6 is a side-view drawing of an alternative embodiment of the invention, including the first portion 602, and two articulating portions, 612 and 614, both of which are hinged, allowing angulation in either direction, as indicated by the arrows. Again, screws such as 620 may be introduced from either side, depending upon the indication.

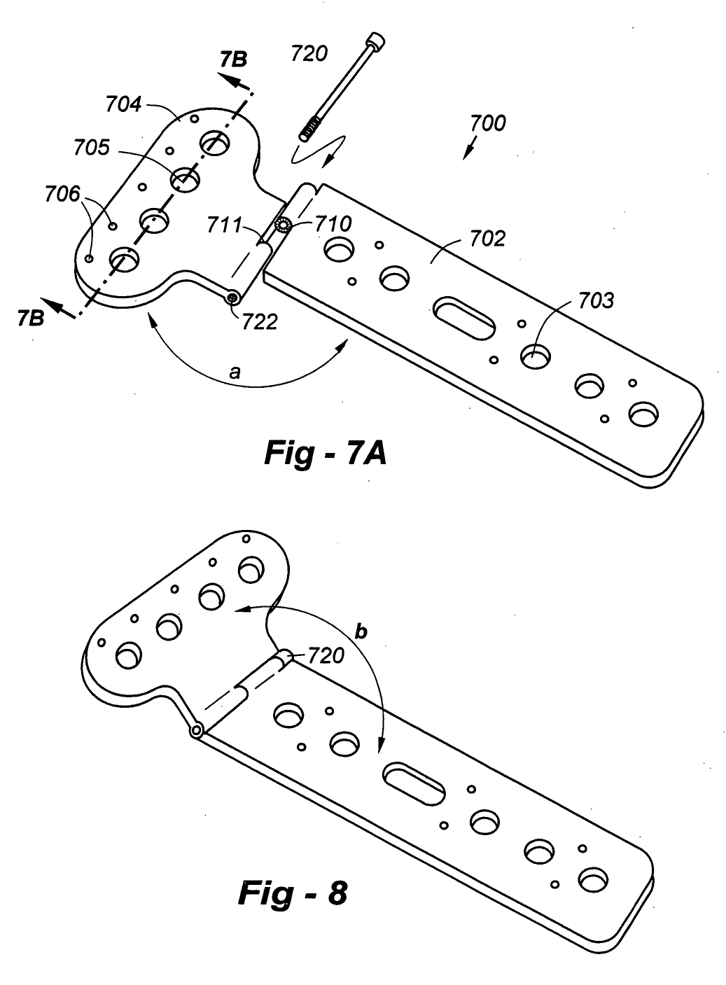

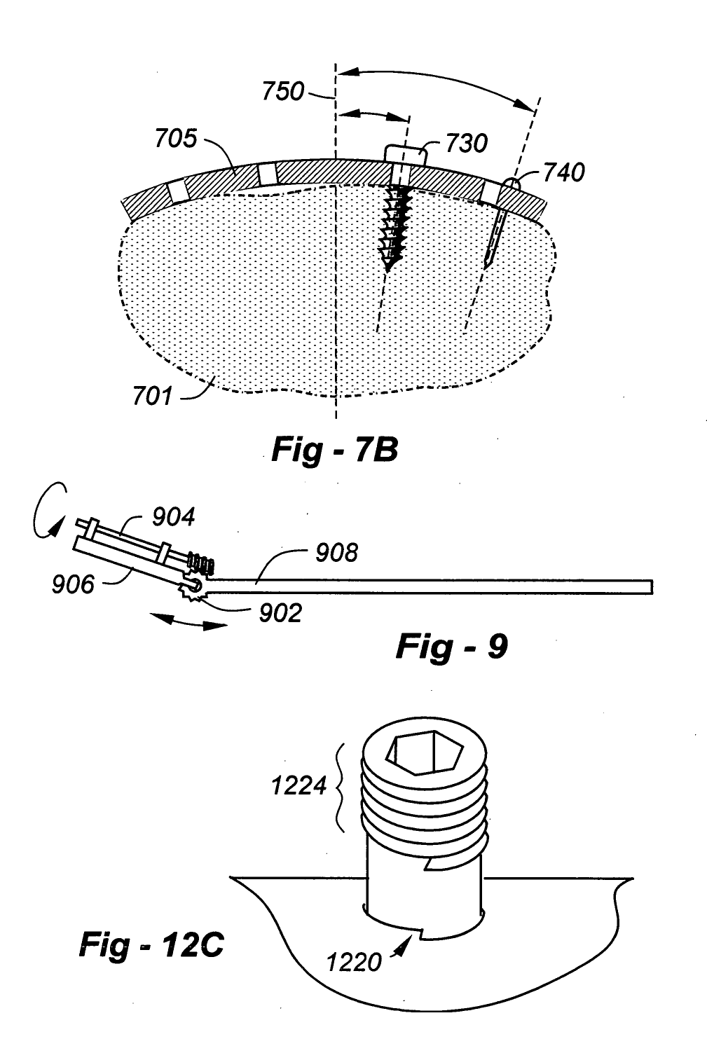

FIG. 7A is a drawing from an open perspective, illustrating a preferred embodiment of the invention particularly suited to distal radius fracture repair. The apparatus, indicated generally at 700, includes a proximal plate portion 702, having screw-receiving slots or holes 703, and a distal portion 704 having one or more screw-receiving slots or holes 705. A distal plate portion 704 may be t-shaped, as shown, which is commonly done for distal radius fixation applications. The crossbar of the T may also form an angle “a” with the proximal portion 702, a geometry which is also known in prior-art devices.

Unique to this invention, however, the device 700 includes an adjustable, lockable fixation between the proximal portion 702 and distal portion 704. In the embodiment of FIG. 7A, this is accomplished through the use of the hinge joint which mates at an interface preferably including roughening or radial grooves 710 and 711. A fastener, such as threaded fastener 720 is inserted through these two hinge portions, to mate with a threaded area 722. As best seen in FIG. 8, screw 720 may be inserted, but left in a loosened state, allowing the angle between the proximally distal portions to be adjusted for a particular patient's physiology, at which time the screw may be tightened, thereby locking the system into position. If multiple articulations are provided, as discussed with reference to FIG. 6, two similar systems may be used for such purpose.

FIG. 7B shows how a portion of plates according to the invention may be curved for greater strength and/or conformity to bone 701. Note that the angle α of screws such as 730 from axis 750 may be different from the angle β for pin insertion for improved fixation. As an alternative to a permanently curved or bent plate, a shape-memory alloy or other material may be used to facilitate insertion in a straightened position, with bending occurring through exposure to body temperature.

FIG. 9 illustrates an alternative mechanism for adjusting the angulation between the two plate portions, namely, the use of a gear 902 and screw drive 904, which, when turned, causes a second plated portion 906 to form a different angle with a first plate portion 908. Once a desired angle is reached, this particular embodiment automatically locks in position, since the gear 902 cannot turn the screw drive 904 to change the angle.

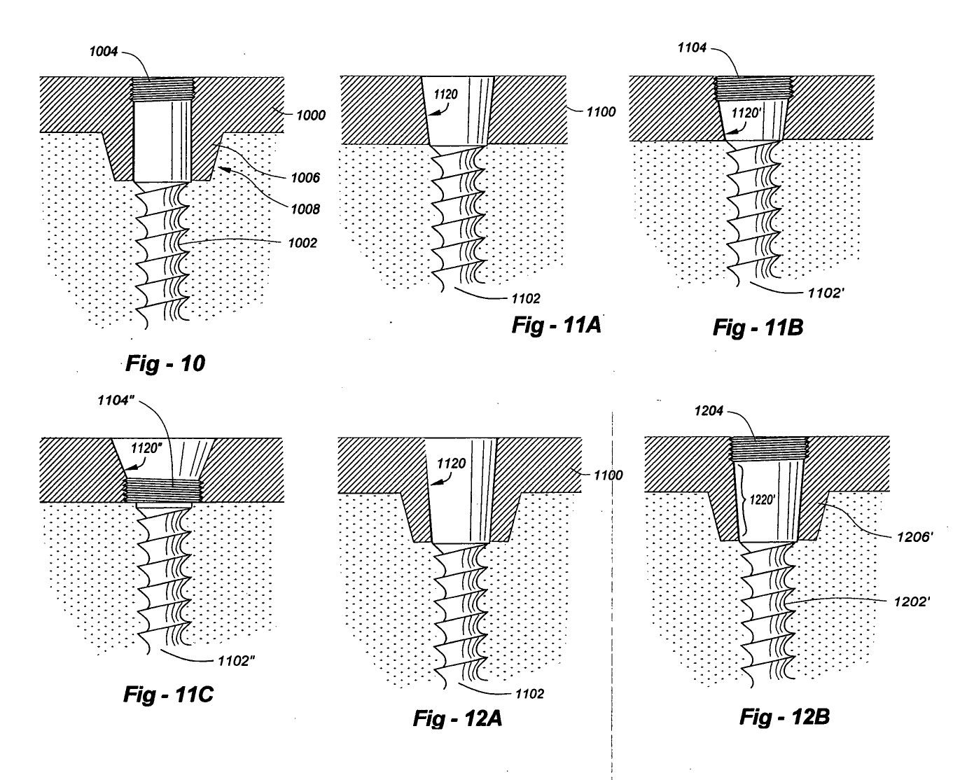

As discussed above, this invention does not preclude the use of poly-axial or locking screws. Indeed, FIGS. 10 through 12 and 15 to 17 illustrate other aspects of this invention which are considered to be patentably distinct, in that such features may be used with the articulating plate systems described herein, or may be adapted to other orthopedic devices and plate systems, including those in current use.

To fortify the area associated with screw placement, FIG. 10 illustrates an improvement in the form of an extended sleeve 1006 around the screw 1002 extending through plate 1000. The use of such a fortification would probably require the formation of a larger hole into the bone, or a counter-sunk area depicted at 1008. This could be accomplished with the inventive stepped drill bit depicted in FIG. 16, which has a larger-diameter proximal portion 1602 and a smaller-diameter distal section 1604. Threaded portion 1004 shows that at least a portion of the proximal shank could be threaded, thereby providing a locking mechanism. However, due to the length and sleeve 1006 through which the screw 1002 extends, a more rigid and substantial screw placement is achieved.

FIGS. 11 and 12 show different improvements, also considered to be patentably distinct. In FIG. 11A, screw 1102 not only includes distal threads, but also includes a proximal tapered portion 1120 that form a metal-metal joint with plate 1100. FIG. 12A shows how this may be used with the extended plate concept introduced with respect to FIG. 10. FIGS. 11B and 12B show how tapered sections may be combined with multiple threaded regions 1102′, 1104′ and 1202′, 1204′. In these embodiments, the plate not only includes locking threads associated with a screw, but in addition, the sides of the screw-receiving aperture and the screw itself are tapered to form a metal-metal joint. Thus, as a user rotates the screw, not only do the threads engage, but a high-integrity tapered metal joint is established in the non-threaded area. The tapered sections 1120, 1220, 1106′, 1206′ depicted here and elsewhere are preferably Morse tapers though other systems may be used. As opposed to a full set of threads in the plates according to this invention, FIG. 12C shows how a tab 1220 may be used instead so long as it cooperates with threads 1224.

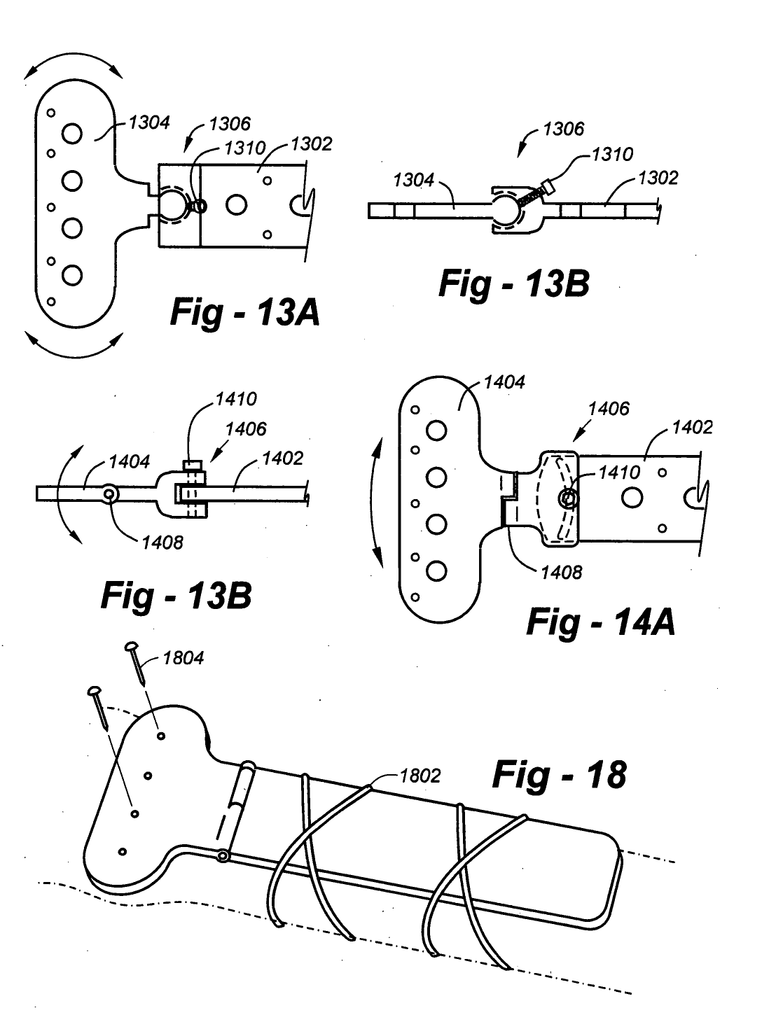

FIG. 13A is an A-P view of an alternative articulation/locking system according to the invention. In this case, plates 1302 and 1304 are coupled through a ball and socket 1306 or other type of joint that allows multiple degrees of freedom before locking the system with fastener 1310. FIG. 13B is a lateral view of the articulation/locking system of FIG. 13A.

FIG. 14A is an A-P view of a different articulation/locking system according to the invention. In this case, plates 1402 and 1404 are coupled through a hinged joint 1408 that allows dorsal and volar flexion, and a rotational joint 1406 that allows radial and ulnar deviation adjustment before locking the system with fastener 1410. FIG. 14B is a lateral view of the articulation/locking system of FIG. 14A.

FIG. 15 illustrates a drill guide according to the invention, which may used in conjunction with any of the plates disclosed herein as well as conventional plate-screw systems and orthopaedic devices. Plate 1502 includes drill guides with extensions 1510 aligned to a patient's physiology. Any of the articulation systems described herein may be provided in region 1520.

FIG. 16 illustrates a patentably distinct stepped drill according to the invention, and FIG. 17 illustrates a patentably distinct stepped screw according to the invention. Screw 1700 includes a tapered section 1702, option non-tapered section 1704 and threaded portion 1706. The proximal end 1708 may include a hex socket, slot, or other fastener-receiving feature. Although the embodiments described herein show the use of screws, circlage cables 1802 or pins 1804 may alternatively be used, alone or in combination.

I claim:

1. A bone plating system adapted for fractures of the distal radius and other indications, comprising:
a first plate portion;
a second plate portion; and
a interconnection between the first and second plate portions enabling the two portions to be adjusted relative to one another and locked into position once a desired relationship is achieved.

2. The bone plate system of claim 1, wherein the first plate portion is generally elongate.

3. The bone plate system of claim 1, wherein the first and second plate portions form a generally T-shaped structure.

4. The bone plate system of claim 1, wherein the interconnection between the first and second plate portions includes a hinge.

5. The bone plate system of claim 1, wherein the interconnection between the first and second plate portions includes a hinge with a frictional interface used to lock the plate portions once the desired relationship is achieved.

6. The bone plate system of claim 1, wherein the interconnection between the first and second plate portions includes a ball-and-socket joint enabling the first and second plate portions to be adjusted in multiple dimensions prior to being locked into position.

7. The bone plate system of claim 1, wherein the first plate portion includes one or more fastener-receiving apertures.

8. The bone plate system of claim 1, wherein the second plate portion includes one or more fastener-receiving apertures.

9. The bone plate system of claim 1, wherein:
the first and second plate portions have a thickness; and
the first, second, or both plate portions include a fastener-receiving aperture having a collar which is longer than the thickness.

10. The bone plate system of claim 1, wherein:
the first and second plate portions have a thickness;
the first, second, or both plate portions include a fastener-receiving aperture having a collar that is longer than the thickness; and
the collar includes an inner surface that forms a tapered locking structure in conjunction with an associated fastener.

11. The bone plate system of claim 1, wherein:
the first, second, or both plate portions include a fastener-receiving aperture adapted to receive a locking screw.

12. The bone plate system of claim 1, wherein the second plate portion is curved.

13. The bone plate system of claim 1, including multiple interconnections between the first and second plate portions, each interconnection enabling the plate portions to be adjusted relative to one another and locked into position once a desired relationship is achieved.

14. The bone plate system of claim 1, wherein the first and second portions are physically separate prior to adjustment and locking, enabling each section to be separately introduced into a recipient as part of a minimally invasive surgical procedure.

15. A bone plating system adapted for fractures of the distal radius and other indications, comprising:
a first plate portion having a plurality of fastener-receiving apertures;
a second plate portion having a plurality of fastener-receiving apertures; and
a interconnection between the first and second plate portions enabling the two portions to be adjusted relative to one another and locked into position once a desired relationship is achieved.

16. The bone plate system of claim 15, wherein the first plate portion is generally elongate.

17. The bone plate system of claim 15, wherein the first and second plate portions form a generally T-shaped structure.

18. The bone plate system of claim 15, wherein the interconnection between the first and second plate portions includes a hinge.

19. The bone plate system of claim 15, wherein the interconnection between the first and second plate portions includes a hinge with a frictional interface used to lock the plate portions once the desired relationship is achieved.

20. The bone plate system of claim 15, wherein the interconnection between the first and second plate portions includes a ball-and-socket joint enabling the first and second plate portions to be adjusted in multiple dimensions prior to being locked into position.

21. The bone plate system of claim 15, wherein:
the first and second plate portions have a thickness; and
the first, second, or both plate portions include a fastener-receiving aperture having a collar which is longer than the thickness.

22. The bone plate system of claim 15, wherein:
the first and second plate portions have a thickness;
the first, second, or both plate portions include a fastener-receiving aperture having a collar that is longer than the thickness; and
the collar includes an inner surface that forms a tapered locking structure in conjunction with an associated fastener.

23. The bone plate system of claim 15, wherein at least one of the fastener-receiving apertures is configured to receive a locking screw.

24. The bone plate system of claim 15, wherein the second plate portion is curved.

25. The bone plate system of claim 15, including multiple interconnections between the first and second plate portions, each interconnection enabling the plate portions to be adjusted relative to one another and locked into position once a desired relationship is achieved.

26. A method of treating a bone fracture, comprising the steps of:
providing the bone plating system of claim 1;
installing the system such that the interconnection between the first and second plate portions is proximate to a fracture site;
adjusting one or both of the plate portions to achieve a desired conformity with the fracture site; and
locking the plates into position once the desired conformity is achieved.

27. The method of claim 26, including a fracture associated with a distal radius.

28. The method of claim 26, further including the steps of:
providing the first and second plate portions as separate units;
separately placing the units into a recipient; and
adjusting one or both of the plate portions to achieve a desired conformity with the fracture site.
